(19) United States
(12) Patent Application Publication
(10) Pub. No.: US 2025/0114316 A1
(43) Pub. Date: Apr. 10, 2025

(54) PLANT-DERIVED NEUROPROTECTIVE COMPOSITION AND A METHOD OF MANUFACTURING THE SAME

(71) Applicant: NBI BIOSCIENCES PVT LTD, Gurugram (IN)

(72) Inventors: Atul KUMAR, Gurugram (IN); Braj Gaurav SHARMA, Mathura (IN)

(21) Appl. No.: 18/687,872

(22) PCT Filed: Aug. 30, 2022
(86) PCT No.: PCT/IN2022/050773

§ 371 Date: Feb. 29, 2024

(30) Foreign Application Priority Data
Aug. 30, 2021 (IN) 202111039282

(51) Int. Cl. A61K 31/198; A61K 9/48; A61K 31/00; A61K 31/191; A61K 31/196; A61K 31/216; A61K 31/336; A61K 31/7076; A61K 36/068; A61K 36/29; A61K 36/31; A61K 36/42; A61K 36/45; A61K 36/48; A61K 36/67; A61K 36/87; A61K 36/8962; A61K 36/899; A61P 25/28

(52) U.S. Cl. CPC A61K 31/198; A61K 9/4858; A61K 31/191; A61K 31/196; A61K 31/216; A61K 31/336; A61K 31/658; A61K 31/7076; A61K 36/068; A61K 36/29; A61K 36/31; A61K 36/3482; A61K 36/42; A61K 36/45; A61K 36/48; A61K 36/67; A61K 36/742; A61K 36/87; A61K 36/8962; A61K 36/899; A61P 25/28

(57) ABSTRACT

The present disclosure relates to a composition comprising an active ingredient or drug for degenerative disorders, one or more plant-derived neuroprotective ingredients and one or more nutraceutically or pharmaceutically acceptable excipient and a method of preparing the same. The composition delivers the active ingredient or the drug to the predetermined location of the body with success.

### FIELD OF THE INVENTION

The invention relates to a composition to plant-derived neuroprotective composition and a method of manufacturing the same.

### BACKGROUND OF THE INVENTION

Parkinson's disease (PD) is one of the most common degenerative disorders of the central nervous system among the elderly characterized by motor symptoms of tremor, rigidity, bradykinesia, and postural instability. The disease is caused by the progressive loss of dopaminergic neurons in the substantia nigra of the midbrain due to which Parkinson's patients cannot synthesize dopamine by themselves and hence has to be supplied externally.

Most of the conventional treatment strategies aim to prevent neuronal loss or protect vulnerable neuronal circuits. The most common treatment for Parkinson's disease is administration of Levodopa (L-DOPA) which is a precursor of dopamine. L-DOPA, unlike dopamine, is able to cross the blood-brain barrier and hence is most commonly used for restoring the dopamine concentration in the brain. However, larger amounts of L-dopa are required to be administered primarily due to short half-life and the conversion of the externally provided L-DOPA to dopamine by the dopamine decarboxylases (DDC). DDC converts the externally supplied L-DOPA to dopamine and since dopamine cannot cross the blood-brain barrier large amounts of L-DOPA are required to be supplied. Moreover, the increase in peripheral dopamine levels leads to irregular heartbeats, nausea, vomiting, anxiety, headache, chills, goosebumps, and shortness of breath.

To overcome the above drawbacks, Carbidopa an inhibitor molecule that inhibits the peripheral dopamine decarboxylase enzyme and limits the conversion of L-DOPA to dopamine has been used traditionally. However, Carbidopa is associated with number of side effects including blurred vision, confusion, agitation, depression or suicidal, fatigue, allergic, dizziness or drowsiness, anxiety etc.

Therefore, there is felt a need for an improved composition which inhibits the peripheral DDC enzymes thereby limiting the conversion of the externally supplied L-DOPA to dopamine. Further, there is a need delivering the active ingredient at the intended region of the body to reduce side effects.

### SUMMARY OF THE INVENTION

In one aspect, the present invention provides a composition comprising an active ingredient or drug for degenerative disorders, one or more plant-derived neuroprotective ingredients and one or more nutraceutically or pharmaceutically acceptable excipient.

In another aspect, the present invention provides a method for preparing the composition.

In still another aspect, the present invention provides a method for the treatment of a patient by delivering the active ingredient or drug for degenerative disorders to a predetermined location, preferably basal ganglia.

In yet another aspect, the present invention relates to use of the composition for delivering an active ingredient or drug for degenerative disorders to a predetermined location in the body, preferably basal ganglia.

### BRIEF DESCRIPTION OF THE DRAWINGS

Reference will be made to embodiments of the invention, examples of which may be illustrated in accompanying figures. These figures are intended to be illustrative, not limiting. Although the invention is generally described in the context of these embodiments, it should be understood that it is not intended to limit the scope of the invention to these particular embodiments.

FIG. 1 shows standard curve of dopamine for DDC assay.

FIG. 2: shows percentage inhibition of different drug molecules on DDC as compared to control.

FIG. 3: shows the saturation i.e., 100% inhibition of the dopa decarboxylase enzyme inhibition by drug sample D (avenanthramide) obtained at 1000 μg/ml confirmed by the 1200 μg/ml.

FIG. 4: shows A. Molecular interactions of Benserazide with Dopamine Decarboxylase enzyme, B. Molecular interactions of phytochemicals with Dopamine Decarboxylase, A1) N-trans-p-Coumarolyl-DOPA.

FIG. 5: shows A. Molecular interaction of Carbidopa with Dopamine Decarboxylase Enzyme, B. Molecular interactions of phytochemicals with Dopamine Decarboxylase, B1) Avenanthramide C, B2) Avenanthramide A, B3) N-trans-p-Coumaroyl-DOPA, B4) 3-O-caffeoyl-D-quini acid, B5) Feruloylquinic acid, B6) Caffeoyl aspartic acid, B7) 4-O-Feryloylquinic acid, B8) 5-Feruloylquinic acid and B9) 4-O-sinapoylquinic acid.

FIG. 6: shows molecular interactions of Dopamine decarboxylase (DDC) enzyme with (a) Cordycepin, (b) Deoxycoriolic acid, and (c) Coriolin

FIG. 7: shows full body PET image (A) Inventive composition (Example 2-(Avenanthramide 70 mg+Chlorogenic acids 30 mg L-dopa 150 mg) (B) Carbidopa (Carbidopa 25 mg+Levodpa 100 mg) (C) Placebo (Carbidopa 25 mg+Levodpa 100 mg) indicating the uptake of 18F-Dopamin by human subject. PET images clearly indicating That the amount of dopamine uptake inside brain (Basal ganglion) in NBI formulation found superior when compared with carbidopa and placebo.

FIG. 8: shows brain PET image (A) Inventive composition (Example 2) (B) Carbidopa (C) Placebo indicating the uptake of 18F-Dopamin by human brain. PET images clearly indicating That the amount of dopamine uptake inside brain (Basal ganglion) in NBI formulation found superior when compared with carbidopa and placebo.

### DETAILED DESCRIPTION OF THE INVENTION

The present invention discloses a composition comprising an active ingredient or drug for degenerative disorders, one or more plant-derived neuroprotective ingredients and one or more nutraceutically or pharmaceutically acceptable excipient.

As used herein, the phrase “active ingredient or drug” refers to those compounds or materials which function as an active pharmaceutical ingredient (API) for veterinary use as well as human pharmaceutical use.

As used herein, the phrase “Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

In an aspect of the present invention is disclosed a composition exhibiting neuroprotective properties. The composition comprises plant-derived neuroprotective ingredients and at least one nutraceutically or pharmaceutically acceptable excipient.

In an embodiment of the present invention the plant-derived neuroprotective ingredients are selected from the group consisting of, but not limited to, levodopa (L-DOPA), avenanthramides, berberine, cordycepin, polysaccharides, adenosine, resveratrol, phytochemicals, epigallocatechin gallate (EGCG), curcumin, peperine and quercetin. Typically, the phytochemicals can be extracted from the plants belonging to the genus Cannabis and Cordyceps. Typically, the phytochemicals can be extracted from the mycelium or fruiting body parts of the plants.

In an embodiment of the present invention the plant-derived neuroprotective ingredients are selected from the extract(s), fraction(s), active compound(s) and phytochemical(s) or mixtures thereof from the group consisting of, but not limited to, Mucuna pruriens, Avena sativa, Berberis aristata, Cordyceps militaris, Cordyceps sinensis, Vitis vinifera, Cannabis indica, Cannabis sativa, Coffea arabica, Curcuma longa, Piper nigrum, Allium cepa, Brassica oleracea and Vaccinium myrtillus. These ingredients are derived from conventional extractions procedures. These ingredients can be the extracts or enriched fractions or pure compounds or the mixtures thereof.

Mucuna pruriens is known commonly as monkey tamarind, velvet bean, Bengal velvet bean, Florida velvet bean, Mauritius velvet bean, Yokohama velvet bean, cowage, cowitch, lacuna bean, and Lyon bean. It is a tropical legume native to Africa and tropical Asia and widely naturalized and cultivated throughout the world. Avena sativa or common oat, is a species of cereal grass known for its highly nutritious seeds. It is common throughout the temperate regions, particularly central India and Eurasia. Berberis aristata is also known as Indian barberry, daru haldi or tree turmeric. It is native to the temperate and sub-tropical regions of Asia, Europe, and America. Cordyceps militaris and Cordyceps sinensis commonly known as caterpillar fungus is native to India, Nepal, Tibet and China but is distributed in humid temperate and tropical forests. Vitis vinifera is the common grape vine which is native to the Mediterranean region, Central Europe, and southwestern Asia, from Morocco and Portugal north to southern Germany and cast to northern Iran. Cannabis indica commonly known as hemp, hashish, marijuana and is native to regions spanning from Southern Europe and Northern Africa to Southwestern Asia and South Asia. Moreover, it has been naturized in many other parts of the world. Coffea arabica also known as the Arabian coffee, coffee shrub of Arabia, mountain coffee or arabica coffee. It is endemic to the southwestern highlands of Ethiopia but widely naturalized in Africa, Latin America, Southeast Asia, China and Caribbean. Curcuma longa commonly known as turmeric or haldi is a perennial, rhizomatous, herbaceous plant native to the Indian subcontinent and Southeast Asia but has become widely naturalized in many parts of the world. Piper nigrum or black pepper or peppercorn are found generally throughout the world and occur primarily in the region spanning Southeast Asia to the Pacific coast of America and other tropical regions.

Typically, the plant extracts are derived from plant material selected from various parts of the plant such as, but not limited to, roots, rhizomes, stems, seeds, barks, flowers, leaves and fruits and is extracted using conventional extraction techniques using conventional solvents. Accordingly, the extracts or the raw materials can be sourced from any of these different natural sources.

The conventional solvents are selected from the group of, but not limited to, water, alcohol, organic solvent and a combination thereof or methods such as cryogenic extraction, maceration, infusion, decoction, percolation, hot continuous extraction (soxhlet), aqueous alcoholic extraction, by fermentation, counter-current extraction, ultrasound extraction (sonication), Cold pressed extraction and supercritical fluid extraction whichever is suitable to obtain complete extract. The extracts may be in solid form or semi-solid form or liquid form or nano-emulsion form.

In an embodiment of the present invention, Mucuna pruriens extract is derived from the seeds of Mucuna pruriens. The seed extract comprises naturally occurring Levodopa which is the precursor of dopamine synthesis. In another embodiment of the present invention, Avena sativa extract is derived from the seeds of Avena sativa. In yet another embodiment of the present invention, Berberis aristata extract is derived from the root, stem, and leaves of Berberis aristata. In yet another embodiment of the present invention, Cordyceps militaris extract is derived from the fruiting body of Cordyceps militaris. The Cordyceps militaris extract comprises cordycepin which inhibits the peripheral dopamine decarboxylase enzyme and facilitates the maximum amount of Levodopa to the brain. Cordycepin also prevents the mitochondrial dysfunction and reduces the oxidative stress in neuronal cells. Cordyceps sinensis extract is derived from the fruiting body of Cordyceps sinensis. In still another embodiment of the present invention, Vitis vinifera extract is derived from the fruit of Vitis vinifera. In yet another embodiment of the present invention, Cannabis indica and Cannabis sativa extract is derived from the flower, leaf, seeds, and fruit of Cannabis indica and Cannabis sativa. In still another embodiment of the present invention, Coffea arabica extract is derived from the seeds of Coffea arabica. The Coffea arabica extract comprises chlorogenic acid (CGA) which is an anti-inflammatory agent and prevents the neurodegeneration in patients suffering from Parkinson's disease. It downregulates the expressions of iNOS, TNF-a, and NF-kB in activated glial cells, thereby inhibiting neuroinflammation through its elevated anti-inflammatory and antioxidant activities. In yet another embodiment of the present invention, Curcuma longa extract is derived from the rhizomes and root of Curcuma longa. In still another embodiment of the present invention, Piper nigrum extract in form of powder is derived from the fruit and seed of Piper nigrum.

The scope of the present invention is not only limited to Mucuna pruriens, Avena sativa, Berberis aristata, Cordyceps militaris, Vitis vinifera, Cannabis indica, Coffea arabica, Curcuma longa and Piper nigrum plants and products derived therefrom but also extends to botanically closely related plants specially belonging to same family, preferably belonging to same genus, still preferably belonging to same species having substantially similar phenotypic and genotypic characteristics.

Typically, in the composition of the present invention, the amount of Mucuna pruriens can be in the range of 5 to 95 wt %, the amount of Avena sativa can be in the range of 5 to 95 wt %, the amount of Berberis aristata can be in the range of 1 to 40 wt %, the amount of Cordyceps militaris can be in the range of 4 to 96 wt %, the amount of Vitis vinifera can be in the range of 5 to 95 wt %, the amount of Cannabis indica and/or Cannabis sativa can be in the range of 1 to 98 wt %, the amount of Coffea arabica can be in the range of 5 to 95 wt %, the amount of Curcuma longa can be in the range of 10 to 90 wt %, the amount of Piper nigrum can be in the range of 15 to 80 wt %, the amount of multi-plant extract can be in the range of 5 to 95 wt % and the amount of at least one nutraceutically or pharmaceutically acceptable excipient can be in the range of 5% to 60% wt %. Typically, the multi-plant extract is selected from at least one of Allium cepa, Brassica oleracea and Vaccinium myrtillus.

In an embodiment of the present invention, at least one nutraceutically or pharmaceutically acceptable excipient is selected from the group consisting of, but not limited to, at least one diluent, at least one super disintegrant, at least one binder, at least one lubricant, at least one glidant and combinations thereof. Typically, the at least one nutraceutically or pharmaceutically acceptable excipient is selected from the group consisting of, but not limited to, Guar gum, hydroxypropyl methylcellulose (HPMC), Microcrystalline cellulose (MCC), Talc, Mg Stearate, Lactose, Cellulose, Polyvinylpyrrolidone (PVP), Isopropyl alcohol (IPA), Propellants such as hydrofluoroalkanes, Methyl cellulose, mannitol, dicalcium phosphate, calcium sulfate, dry starch, cellulose, kaolin, sodium chloride, anhydrous lactose, sorbitol, sucrose, Polyethylene glycol (PEG), Polyoxymethylene stearates and Lauryl sulphate salts.

The composition of the present invention may be in the form of, but not limited to, a semi-solid mass, powder, an oil and water-soluble dispersion, nano emulsion, a capsule, tablet, syrup, a blend, a suspension, nasal drop or spray, dry powder, granules and the like. In an exemplary embodiment of the present invention the composition of the present invention can be encapsulated and in a dosage form of a capsule. Depending upon the dosage the composition may comprise further excipients necessary for the manufacture of the preferred dosage form and its breakdown following ingestion. In an embodiment of the present invention the composition may comprise one or more active ingredients selected from vitamins, minerals, phytochemicals, antioxidants, and combinations thereof. In another embodiment of the present invention, the composition may further comprise one or more fillers with neuroprotective ability.

In a preferred embodiment, the active ingredient or drug is in the range of 10 mg to 500 mg, the plant-derived neuroprotective ingredients is in the range of 50 mg to 300 mg and the nutraceutically or pharmaceutically acceptable excipient is in the range of 1% to 20%.

In a preferred embodiment, the composition additionally comprises one or more active ingredients selected from vitamins, minerals, antioxidants, Omega, and trace elements and combinations thereof.

In another aspect, the present invention discloses a method for preparing the composition.

In an embodiment, the method of preparation comprises the following steps:
proportioning and weighing the neuroprotective ingredients and converting into the powder;
sieving in through a pre-determined mesh size;
blending the superfine powder with the drug for degenerative disorders and one or more nutraceutically or pharmaceutically acceptable excipient; and
filling the homogenous blend of active ingredients and excipients in the enteric coated capsule.

Typically, the method involves proportioning and weighing the ingredients and converting into the superfine powder followed by sieving in a sieve having a pre-determined mesh size. The superfine powder then undergoes blending to obtain the composition of the present invention. Typically, the wet ingredients such as CBD oil are converted into the dry powder form using the adsorbent such as Nuslin/Aerosil during blending.

In a preferred embodiment, the mesh size is in the range of 50 to 250. Typically, the mesh size is 100/200.

As used herein, the phrase “superfine powder” refers to homogenous blend or pharmaceutically accepted free flow homogenous powder.

The composition of the present invention may be in the form of, but not limited to, enteric-coated tablet by direct compression or wet granulation methodology, capsule in the dry state, aerosols for inhalation or nasal drops, oral syrup, effervescent granules powder and topical solution or ointment, dermal delivery. In an exemplary embodiment of the present invention the composition of the present invention can be encapsulated and in a dosage form of a capsule. Depending upon the dosage the composition may comprise further excipients necessary for the manufacture of the preferred dosage form and its breakdown following ingestion.

In another aspect, the present invention discloses a method for the treatment of a patient by delivering an active ingredient or drug for degenerative disorders, to a predetermined location of the body.

In an embodiment of the invention, predetermined location of the body is basal ganglia.

In another aspect, is disclosed the use of the composition the present invention for delivering an active ingredient or drug for degenerative disorders to a predetermined location in the body, preferably basal ganglia.

In another aspect, is disclosed the use of the composition for improving the gut permeability.

In another aspect, is disclosed the use of the composition for inhibiting the coagulation of α-synuclien protein.

Advantageously, the composition of the present invention exhibits the below mentioned properties:
Prevents the α-synuclein aggregation inside the gut & brain and improves the gut barrier (Intestinal permeability) and stops the entry of undesired/infectious agents such as chemo toxicant and microbes into the blood & brain.
Prevents dopamine degradation by enzymes and microbes present in inside gut;
Enhances the bioavailability of Dopamine to the brain by increasing the bioavailability of L-Dopa in the brain by inhibiting the peripheral DDC enzyme activity.
Improves the mitochondrial dysfunction and the chronic fatigue by preventing or reducing mitochondrial dysfunction; and
regenerates the dopamine producing neurons in substantia nigra and the basal ganglion by reversing cell injury by repressing Endoplasmic Reticulum stress (ER-stress), by dysregulation of micro-RNA (miR-7) and SNCA gene (Encoding Alfa-synuclein protein) and by improving mitochondrial function.

The composition of the present disclosure comprises drugs that are useful for, but not limited to, degenerative disorders selected from Parkinson's disease (PD), Alzheimer, chronic depression, Transient ischemic stroke, Dementia, Epilepsy and Ataxia.

### EXAMPLE

The following examples are illustrative of the invention but not limitative of the scope thereof:

### Materials and Methods

### Materials

Carbidopa was obtained from Merck (US grade standard).

Benserazide was obtained from Merck (European pharmaceutical standard grade).

Phytochemicals (N-trans-p-coumarolyl, Levodopa, Serine, Aventhramide C, Aventhramide A, 3-O-caffeoyl-D-quinic acid, feruloylquinic acid, caffeoyl aspartic acid, 4-O-Feruloylquinic acid, 5-feruloylquinic acid, 4-O-sinapolyquinic acid) were obtained from Avena sativa, Berberis aristate, Cordyceps militaris, Vitis vinifera, Cannabis indica, Cannabis sativa, Coffea arabica, Curcuma longa from India.

In vitro screening of dopamine decarboxylase (DDC) inhibition potential and MTT assay status of plant-derived neuroprotective ingredients.

The in vitro screening was conducted to assess the plant-derived neuroprotective ingredients for their cytotoxic potential, selectivity as potent inhibitor for dopamine decarboxylases (DDC) enzyme and effectiveness in inhibiting the peripheral DDC activity without altering the function of other pyridoxal phosphate enzymes (PLP enzymes).

MTT assay: Cytotoxic potential of eight different compounds (A, B1, B2, C1, C2, D1, D2 and D3) were determined and the assay was performed on five different cell lines viz: A549 (human lung carcinoma cell line), MCF (Human breast cancer cell line) HCAT (human keratinocytes cells), HCT 116 (human colon carcinoma) and N2A (mouse neuroblastoma cell line).

Cells were plated at a density of 10,000 cells per well in a 96 microplate. After 24 h cells were treated with different concentrations of compounds A (1% Avenanthramide), B1, B2 (5% Cordycepin) C1, C2 (60% Chlorogenic acid), D1, D2 and D3 (98% Avenanthramide) and incubated for different time intervals. After 24 h and 42 h of incubation, 3-[4,5-dimethylthiazol-2-yl]-2,5-diphenyl tetrazolium bromide (MTT) solution (0.5 mg/ml in MEM) was added to 96 well plate, and then cells were incubated for 4 h at 37° C. Then the media with MTT were removed and the purple formazan crystals formed were solubilised using 100 μL DMSO and the absorbance were measured at 570 nm using microplate reader (EnVision Multimode Microplate Reader, Perkin Elmer, USA). The summary of results is shown in Table 1. Film forming compounds were not selected for the DDC assay.

TABLE 1
Summary of results of cytotoxic potential different compounds

| Cell line | | | | | | | | |
|---|---|---|---|---|---|---|---|---|
| name | A1 | B1 | B2 | C1 | C2 | D1 | D2 | D3 |
| A549 | Not toxic upto | Not toxic upto | Not toxic upto | Not toxic | Not toxic upto | Not toxic upto | Not toxic upto | Not toxic upto |
| IC50 | 10-fold dilution | 82.5 μg/ml | 1000 μg/ml | 1000 μg/ml | [symbol] μg/ml | [symbol] μg/ml | [symbol] μg/ml | 250 μg/ml |
| (μg/ml) | NA | 195.4 | NA | NA | | [symbol] | [symbol] | 334.3 |
| HCT116 | Not toxic upto | Not toxic upto | Not toxic upto | Not toxic upto | Not toxic upto | Not toxic upto | Not toxic upto | Not toxic upto |
| IC50 | 5-fold dilution | 1000 μg/ml | 1000 μg/ml | 1000 μg/ml | [symbol] μg/ml | 12.5 μg/ml | 100 μg/ml | 12.5 μg/ml |
| (μg/ml) | NA | NA | NA | NA | [symbol] | 38.28 | 143.7 | 26.95 |
| MCF7 | Not toxic upto | Not toxic upto | Not toxic upto | Not toxic upto | Not toxic upto | Not toxic upto | Not toxic upto | Not toxic upto |
| IC50 | 10-fold dilution | [symbol] μg/ml | [symbol] μg/ml | 1000 μg/ml | [symbol] μg/ml | 3.9 μg/ml | 100 7.89 μg/ml | 7.89 μg/ml |
| (μg/ml) | NA | 30.35 | [symbol] | NA | 22.2 | 82.5 | [symbol] | 83.18 |
| HACAT | Not toxic upto | Not toxic upto | Not toxic upto | Not toxic upto | Not toxic upto | Not toxic upto | Not toxic upto | Not toxic upto |
| IC50 | 10-fold dilution | 1000 μg/ml | 1000 μg/ml | 500 μg/ml | [symbol] μg/ml | 62.5 μg/ml | 100 62.5 μg/ml | 500 μg/ml |
| (μg/ml) | NA | NA | NA | [symbol] | 21.13 | 142.5 | [symbol] | NA |
| N2A | Not toxic upto | Not toxic upto | Not toxic upto | Not toxic upto | Not toxic upto | | | |
| IC50 | 10-fold dilution | 1000 μg/ml | 1000 μg/ml | 500 μg/ml | [symbol] μg/ml | | | |

| | | | | | |
|---|---|---|---|---|---|
| (μg/ml) | Drug producing a | [symbol] | [symbol] | 173 | Drug producing a layer on cells |
| | layer on cells. | | | | |

[symbol]

indicates data missing or illegible when filed

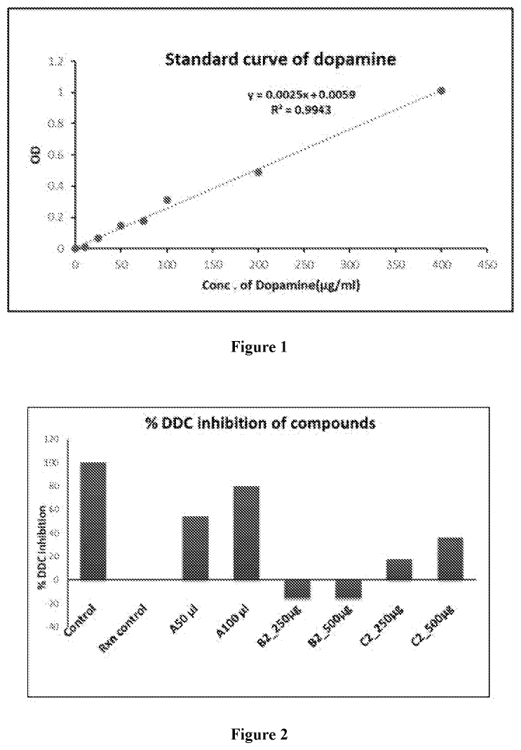

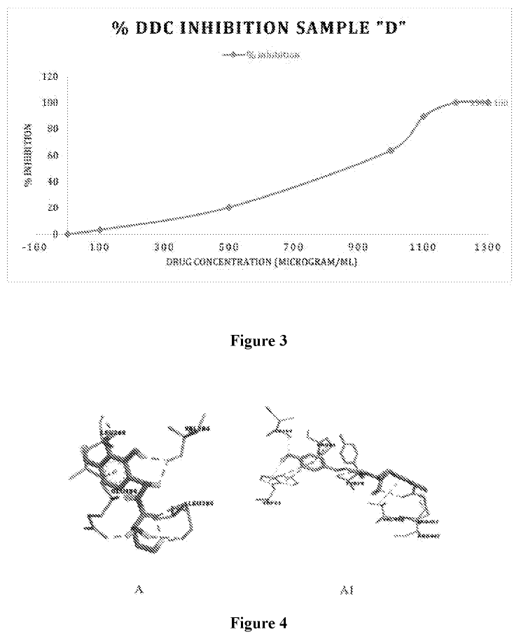

Dopa Decarboxylase (DDC) enzyme inhibition assay: The DDC activity of selected compounds (A1, Bland C1) based on MTT assay were evaluated by their ability to convert the substrate 3,4-dihydroxy-L-phenylalanine (L-Dopa) to 3,4-dihydroxyphenylethylamine (dopamine). The dopamine product is measured by its absorbance at 340 nm after derivatization with trinitrobenzene sulfonic acid. Sherald, F. et al. (1973) and Charteris and John, 1975. The standard curve of dopamine was also plotted to determine the linear range of the assay (FIG. 1).

The assay was performed by using standard method available. Briefly, 150 μl of 5 ng/μL rhDopa (Recombinant Human Dopa Decarboxylase) mixed with 10 μL of 20 mM L-DOPA+4 μL of 5 mM pyridoxal phosphate+36 μL assay buffer reaction mixture was incubated at 37° C. for 30 minutes. The control was kept without enzyme. The enzyme was preincubated with respective drug concentrations in 100 μL as mentioned in Table 2 (Mixture compounds) and Table 3 (Isolated compound) for 10 mins before adding substrate and cofactor. The total reaction volume was 300 μL. The reaction stopped by heating for 2 minutes at 95-100° C. After heating, the reaction mixture was cooled on ice and added 10 μL of 5% TNBS to each tube and vortexed. In a fume hood, 300 μL of benzene was added to each reaction and vortexed for 30 seconds. The reaction mixture was centrifuged for 5 minutes to separate the aqueous and organic layer. The benzene phase (the upper layer) of each reaction was pipetted out (200 μl) and loaded into a UV transparent microplate. Microplate was kept covered as much as possible to minimize evaporation of the benzene and read at an absorbance of 340 nm in an endpoint mode and the % inhibition was calculated by comparing with the control group. The results are shown in Table 2, Table 3 and FIG. 2.

TABLE 2
Percentage inhibition of different drug
molecules on DDC as compared to control

| Sample | % Inhibition |
|---|---|
| Control (L-Dopa without enzyme) | 100 |
| Rxn Control (L-Dopa with enzyme) | 0 |
| A50 μl | 55 |
| A100 μl | 80 |
| B2_250 μg (1000 μg/ml) | −15 |
| B2_500 μg (2000 μg/ml) | −16 |
| C2_250 μg (1000 μg/ml) | 18 |
| C2_500 μg (2000 μg/ml) | 36 |

TABLE 3
Percentage inhibition isolated drug molecules (Avenanthramide
with different concentration) on DDC as compared to control.

| Sample | % inhibition |
|---|---|
| Control | 100 |
| rxn control | 0 |
| D1300 | 100 |
| D1200 | 100 |
| D1100 | 89.4 |
| D1000 | 63.6 |
| D500 | 20.4 |
| D100 | 3.2 |

*D 1000 (1000 μg/ml), D 500 (500 μg/ml), D 100 (100 μg/ml)

In silico screening of Dopamine decarboxylase (DDC) enzyme to identify plant-derived neuroprotective ingredients and their inhibition potential was conducted. Plant-derived neuroprotective ingredients or phytochemicals following the Lipinski rule and also not crossing the blood brain barrier (BBB) were carried for further study.

Three phytochemical derivatives of Benserazide and nine phytochemical derivatives of Carbidopa and were docked along with the respective inhibitors of Dopamine decarboxylase enzyme using autodock tool. The blood brain barrier (BBB) results, binding energy, inhibition constant and active interacting residues are provided in Table 4 and Table 5 respectively.

TABLE 4
Analogues list of Benserazide

| | | | | Binding | | |
|---|---|---|---|---|---|---|
| | | | | Energy | Inhibition | Active |
| | | | BBB | kcal/ | Constant | Interacting |
| S.No. | Name | Structure | Prediction | mol | (uM) | Residues |
| Drug | Benserazide | 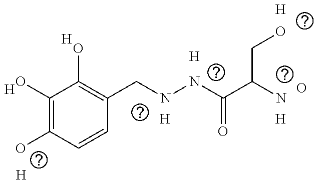 | Negative | −4.78 | 315.24 | VAL204, LEU200, LEU206, GLU196 |

Phytochemicals

| | | | | | | |
|---|---|---|---|---|---|---|
| 1 | N-trans-p- Coumaroyl- DOPA | 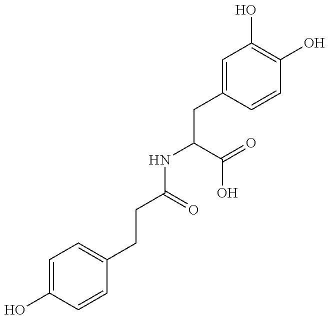 [SMILES: O=C(CCc1ccc(O)cc1)NC(Cc1ccc(O)c(O)c1)C(=O)O] | Negative | −7.43 | 3.59 | ARG447, TYR79, PRO81, TRP71, ILE101, VAL436, PRO437 |
| 2 | Levodopa | 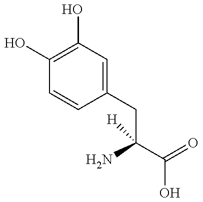 [SMILES: N[C@@H](Cc1ccc(O)c(O)c1)C(=O)O] | Negative | −5.59 | 79.59 | ARG358, GLU125, VAL143, ARG355, CYS111, ILE101, ILE144 |
| 3 | Serine | 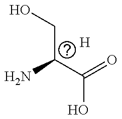 | Negative | −5.09 | 186.11 | ARG355, ILE144, GLU115, ARG358 |

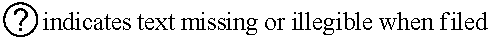

Molecular interactions of Benserazide and phytochemicals with Dopamine decarboxylase (DDC) enzyme is provided in FIG. 4.

TABLE 5
Analogues list of Carbidopa

| | | | | Binding | Inhi- | |
|---|---|---|---|---|---|---|
| | | | BBB | Energy | bition | Active |
| | | | Pre- | kcal/ | Constant | Interacting |
| S.No. | Name | Structure | diction | mol | (μM) | Residues |
| Drug | Carbidopa | 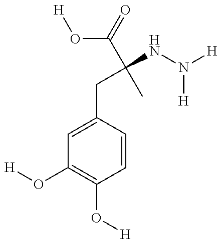 [SMILES: C[C@@](Cc1ccc(O)c(O)c1)(NN)C(=O)O] | Negative | −5.27 | 137.03 | ARG447, TYR79, LYS303, THR246 |

Phytochemicals

| | | | | | | |
|---|---|---|---|---|---|---|
| 1 | Avenanthramide C | 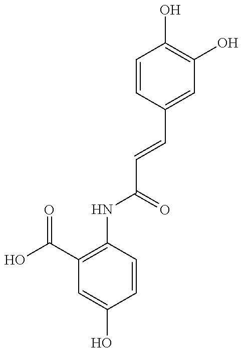 [SMILES: O=C(/C=C/c1ccc(O)c(O)c1)Nc1ccc(O)cc1C(=O)O] | Negative | −7.94 | 1.52 | ARG 447, ASPB27, TYRB79, LYSB303, TYRB27, ALAB148, THRB246, HISB19 |
| 2 | Avenanthramide A | 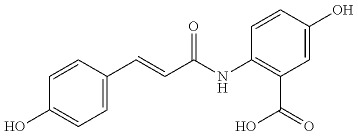 [SMILES: O=C(/C=C/c1ccc(O)cc1)Nc1ccc(O)cc1C(=O)O] | Negative | −7.65 | 2.48 | ALAB148, ALAB273, HISB192, ASPB271, LYSB303, THRB246, ARGB447, TYRB79 |
| 3 | N-trans-p- Coumaroyl- DOPA | 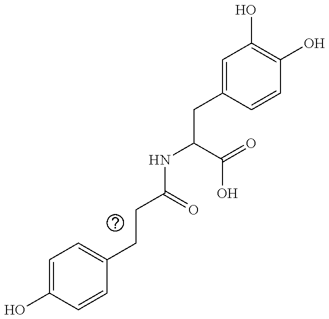 [SMILES: O=C(CCc1ccc(O)cc1)NC(Cc1ccc(O)c(O)c1)C(=O)O] | Negative | −7.30 | 4.43 | ASP92, ASPB92, LYSB361, MET89, CYS95, ARG356, GLY99 |
| 4 | 3-O-caffeoyl- D-quinic acid | 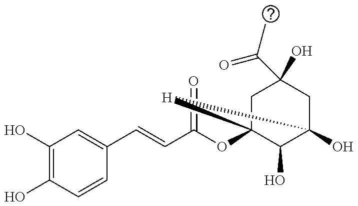 | Negative | −6.18 | 29.71 | ARGB356, SERB108, SERB104, THRB116, THRB112 |
| 5 | Feruloyl quinic acid | 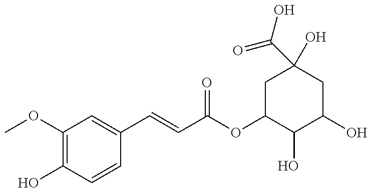 [SMILES: COc1cc(/C=C/C(=O)OC2CC(O)(C(=O)O)CC(O)C2O)ccc1O] | Negative | −5.96 | 42.43 | ASNB132, GLYB142, GLUB115, ARGB358, THRB116, THRB112, ARGB356 |
| 6 | Caffeoyl aspartic acid | 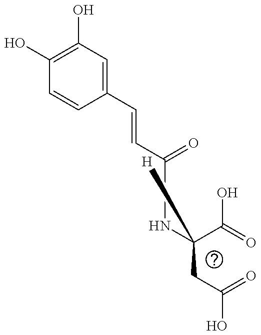 [SMILES: O=C(O)C[C@H](NC(=O)/C=C/c1ccc(O)c(O)c1)C(=O)O] | Negative | −5.91 | 46.75 | ARG 447, TYR30, ALA78, PHE76, TRP71 |
| 7 | 4-O- Feruloylquinic acid | 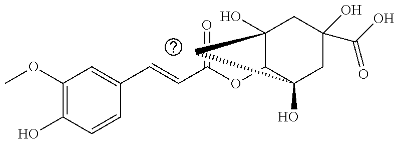 [SMILES: COc1cc(/C=C/C(=O)OC2[C@]3(O)CC(O)(C(=O)O)C[C@]2(O)C3)ccc1O] | Negative | −5.70 | 66.57 | METB89, MET93, CYSB95, PHEB357, MET89, ASN308, GYYB99, METB93 |
| 8 | 5-Feruloylquinic acid | 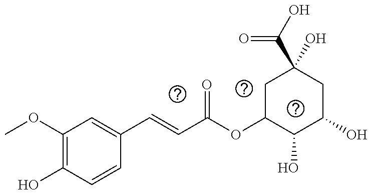 [SMILES: COc1cc(/C=C/C(=O)OC2C[C@@](O)(C(=O)O)C[C@H](O)[C@@H]2O)ccc1O] | Negative | −5.59 | 80.28 | METB89, GLY99, METB93, ASPB92, CYS95 |
| 9 | 4-O- sinapoylquinic acid | 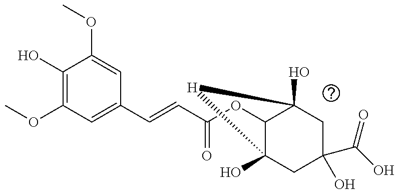 | Negative | −5.54 | 87.62 | ARG447, ALA78, ASN300, ALA148, SER149, SER147 |

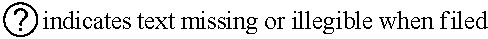

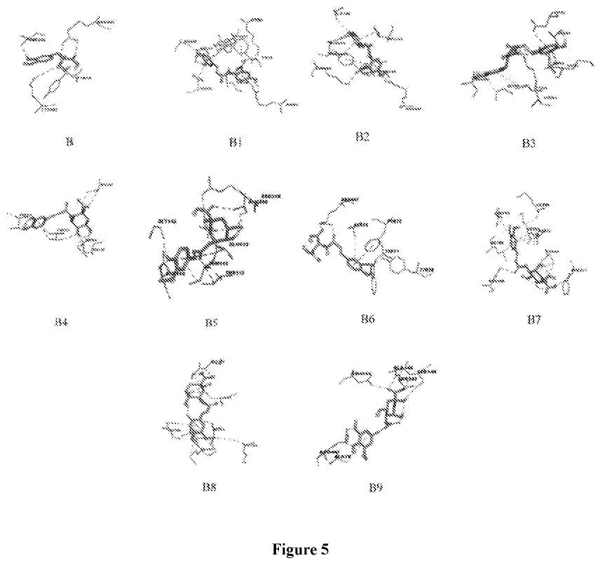

Molecular interactions of Carbidopa and phytochemicals with Dopamine decarboxylase (DDC) enzyme is provided in FIG. 5.

Based on the comparative analysis it was found that Avenanthramide C. Avenanthramide A. N-trans-p-Coumaroyl-DOPA. 3-O-caffeoyl-D-quinic acid. Feruloylquinic acid. Caffeoyl aspartic acid, 4-O-Ferulyolquinic acid, 5-Feruloylquinic acid and 4-O-sinapolylquinic acid phytochemicals have binding energy between 5.5-7.94 kcal/mol. Mentioned molecules have the lowest binding affinity and lower inhibition constant as compared to inhibitor molecule Carbidopa i.e. −5.27 kcal/mol (refer to Table 5 and FIG. 5).

On the other hand, phytochemicals N-trans-p-Coumaroyl-DOPA, Levodopa and serine were found to have binding energy between 5-7.43 kcal/mol. Mentioned molecules have the lowest binding affinity and lower inhibition constant as compared to inhibitor molecule Benserazide i.e. −4.78 (refer to Table 4 and FIG. 4).

In silico screening of Dopamine decarboxylase (DDC) enzyme to identify inhibitor molecules from mushroom was conducted.

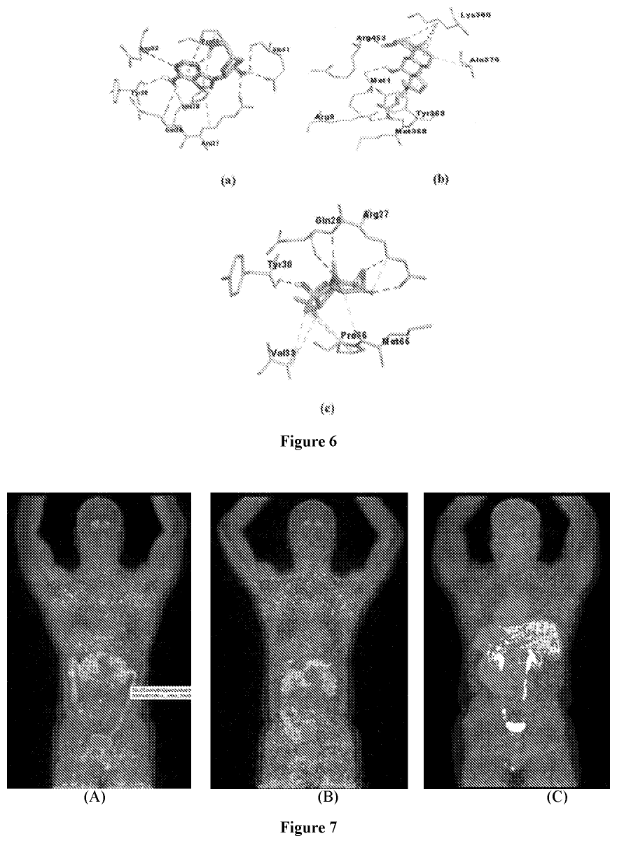

Four active phytochemicals of Mushroom (Cordycepin, Coriolin, Deoxycoriolic acid and cordycepic acid) were evaluated. By applying drug-likeness screening BBB penetration studies only three (Cordycepin, Coriolin and Deoxycoriolic acid) showed BBB negative, but applying physicochemical properties only one is found BBB negative. Docketing studies on the three phytochemicals were done and Deoxycoriolic acid was found to have the lowest binding energy and inhibition constant. The blood brain barrier (BBB) results, binding energy, inhibition constant and active interacting residues are provided in Table 6 and FIG. 6.

TABLE 6
Binding Energy (kcal/mol), Inhibition constant (μm), and active interacting
residues of phytochemicals curated from Mushroom.

| | | | BBB | | | |
|---|---|---|---|---|---|---|
| | | | Penetration | Binding | | |
| | BBB | | (Physico- | Energy | Inhibition | Active |
| Phyto- | Penetration | | chemical | (kcal/ | Constant | Interacting |
| chemicals | (Prediction) | Structure | properties) | mol) | (μM) | Residues |
| Cor- dycepin | Negative | 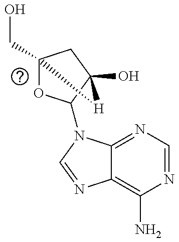 | Negative | −4.52 | 487.85 | ASP32, PRO66, GLU61, HIS70, TYR30, GLN28, ARG27 |
| Deoxy- coriolic acid | Negative | 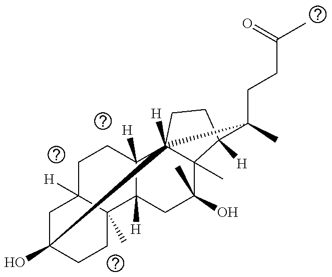 | Positive | −7.16 | 5.61 | ARG453, LYS380, ALA376, MET1, ARG9 TYR369, MET368 |
| Coriolin | Negative | 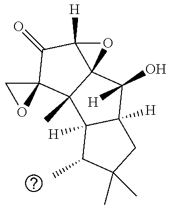 | Positive | −6.45 | 18.68 | TYR30, ARG27, GLN28, PRO66, VAL33, MET65 |

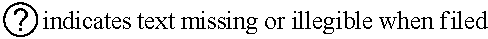

### Method for the Preparation of the Composition According to Present Invention

Example 1 (NBIPAR07A): Accurately weighing of active ingredients and excipients as per the calculation of 1000 capsules. Specifically, with the avenanthramide 100 mg+150 mg L-dopa per capsule. In-process quality control of individual material. Blending for 60 minutes at 90 rpm and filling the blend to prepare the capsule.

Example 2 (NBIPAR07B): Accurately weighing of active ingredients and excipients as per the calculation of 1000 capsules. Specifically, with the avenanthramide 70 mg+30 mg chlorogenic acid+150 mg L-dopa per capsule. In-process quality control of individual material. Blending for 60 minutes at 90 rpm and filling the blend to prepare the capsule.

Example 3 (Carbidopa): “Syndopa” a product of Sun Pharma was used.

Example 4 (Placebo): Accurately weighing of active ingredients and excipients as per the calculation of 1000 capsules. Specifically, with the inert lactose 100 mg+150 mg L-dopa per capsule. In-process quality control of individual material. Blending for 60 minutes at 90 rpm and filling the blend to prepare the capsule.

A comparative clinical study to evaluate the DDC inhibition efficacy of Example 1 and Example 2 with marketed carbidopa-based formulation & placebo in six adult human subjects were conducted to evaluate the DDC enzyme inhibition efficacy of composition (using F-Dopa imaging). Further, the release profile of composition inside GIT through gamma scintigraphy (^{99m}TcO4) and the safety of the subjects were also studied.

### Study Design:

TABLE 7

| Parameter | Credential |
|---|---|
| Study Type | Interventional |
| Study phase | Academic Clinical Study (ACS) |
| Blinding | Open Label |
| Centre | Single centre |
| Recruitment | ICF |
| Endpoint Classification | Efficacy Study |
| Period | Single period, cross over |
| Treatment | Single treatment, cross over |

Study population and sample size: Six healthy adult human subjects.
Housing: Two hours prior to at least 2 hours post dosing
Radioactive element used for radiolabeling: 18F & Technetium Pertechnetate—^{99m}TCO₄
Radiation Dose: 100-500 μCi of Technetium Pertechnetate per Capsule/Capsule

Inclusion Criteria: The following criteria was utilized:
i. Subjects with clinically suspected parkinsonian features, including but not limited to following constellation of symptoms:
   Tremor: can occur at rest, in the hands, limbs, or can be postural
   Muscular: stiff muscles, difficulty standing, difficulty walking, difficulty with bodily movements, involuntary movements, muscle rigidity, problems with coordination, rhythmic muscle contractions, slow bodily movement, or slow shuffling gait
   Sleep: early awakening, nightmares, restless sleep, or sleep disturbances
   Whole body: fatigue, dizziness, poor balance, or restlessness
   Cognitive: amnesia, confusion in the evening hours, dementia, or difficulty thinking and understanding
   Speech: difficulty speaking, soft speech, or voice box spasms
   Nasal: distorted sense of smell or loss of smell
   Urinary: dribbling of urine or leaking of urine
   Mood: anxiety or apathy
   Facial: jaw stiffness or reduced facial expression
   Also common: blank stare, constipation, depression, difficulty swallowing, drooling, falling, fear of falling, loss in contrast sensitivity, neck tightness, small handwriting, trembling, unintentional writhing, or weight loss
ii. Subject who are able to understand and ready to provide written informed consent.
iii. Subject must be male human beings greater than 18 years of age.
iv. Subject should be having Body Mass Index (BMI) in the range 18.5-30 kg/m2 and weighing at least 50 kg.
v. Subject's health status as determined by medical history and physical examination, ECG and laboratory tests performed within 21 days prior to the commencement of the study.
Subject whose screening laboratory values are within normal limits or considered by the physician/Principal Investigator to be of no clinical significance.

Exclusion Criteria: The following criteria was utilized:
i. Subject incapable of understanding the informed consent process or not ready to sign informed consent.
ii. Subject with significant history of hypersensitivity to Study Drug or any ingredients of the formulation or any related products as well as severe hypersensitivity reactions (like angioedema) to any drugs.
iii. Subject with of presence or history of significant gastrointestinal, liver or kidney disease, or any conditions known to interfere with the absorption, distribution, metabolism or excretion of drugs or known to potentiate or predispose to undesired effects.
iv. Subject with active peptic ulceration or a history of peptic ulceration.
v. Subject with resting hypotension (BP<90/60) or hypertension (BP>139/89).
vi. Subject with Pulse rate below 50/min. and above 99/min.
vii. Subjects with or prior history of clinically significant, Cardiovascular, pulmonary, hepatic, renal, hematological, gastrointestinal, endocrine, immunologic, dermatologic musculoskeletal, neurological or psychiatric disease.
viii. Investigations with urine samples of subject's shows clinically abnormal chemical and microscopic examination of urine defined as presence of RBC, WBC, >4HPF, Glucose (Positive) or Protein (Positive).
ix. Subjects with a history of MI, Stroke, Peripheral Arterial Disease, GI Bleeding, Hepatic-Impairment, Renal Impairment, Epilepsy and Intracranial hemorrhage.
x. Subject has inability to communicate well (i.e. language problem, poor mental development, psychiatric illness or poor cerebral function) that may impair the ability to provide, written as well as audio-video informed consent.
xi. Subject with a history of known food allergy.
xii. Subject who have suffered any illness or who have been hospitalized within the last 4 weeks preceding the start of the study.
xiii. Subject who have taken over the counter or prescribed medications, including any enzyme modifying drugs within the last 14 days prior to the study.
xiv. Subject with a history of drug abuse or alcoholism i.e. alcohol consumption >2 units/day or 10 units/week (one unit of alcohol=50 ml spirit or 200 ml wine or 500 ml beer).
xv. Subject with smoking history of >10 Cigarettes/day or Tobacco consumption >4 packets/day.
xvi. Subject who was participated in any other clinical trial requiring repeated blood sampling or a blood donation program or blood loss of more than 450 ml, in the past three months (approx. 90 days) (This 450 mL includes the total blood loss that will occur during the study).
xvii. Subject with clinically significant abnormal lab values.
xviii. Subject with positive Breath Alcohol Analysis before admission.

A unique ID (NBIPAR01 to NBIPAR06) was allotted to each study subject to maintain their identity confidential.

The radiolabelled formulation was given to the subjects in the supine condition with 250 ml of safe drinking water. The radiolabelled formulation was monitored to examine its release in the upper part of GIT. The static images of the abdomen was acquired under the gamma camera at an interval of 30 min starting from oral ingestion at time t=0. The release time/Capsule bursting time was noted. Drinking of water was restricted from at least 01 hour prior to dosing until at least 01-hour post-dose, (except for water given with dose administration). Subjects was not consuming alcohol and smoke 48 hours before drug administration and throughout study period. Subject was not consumed grapefruit containing products for 48 hours before the drug administration and throughout the study. The food to the subject was allowed after the whole-body imaging or completion of study/imaging. Subjects were maintained in semi recumbent position on bed for first 08 hours post-dose and only necessary movement was allowed during that period. Thereafter subject was free to ambulate for remainder of the study period.

Following the scintigraphy based capsule releasing time, 18F-DOPA was injected into the same subject within 60 minutes of capsule release/drug release. Whole-body and dedicated Brain PET images was acquired as per standard protocol and parameters. 18F-DOPA uptake was quantified in the different body organs such as the Brain (Basal ganglia and cerebellum), Liver, muscles and mediastinal blood pool etc. In brief the 18F-Dopa was monitored in the Basal ganglia (Left & Right), Thalamus (Left & Right), Cerebellum (Left & Right), Parietal (Left & Right), Blood Pool, Liver, Spleen, Pancreases, Renal cortex and Renal pelvis.

Results: The capsule (Example 1 and Example 2) bursting & release time inside the GIT in six healthy human subjects was observed and found that the capsule released the drug in small intestine at the time of 2 hours±0.3 Hr. There was no evidence of drug release in the stomach indicating the effectiveness of capsule to bypass stomach without release.

18F-DOPA PET study: In order to assess extent of DOPA uptake in various organs of brain and whole body region, semiquantitative analysis was performed by obtaining standardized uptake value i.e. SUV Max, SUV Mean and SUV Minimum values by drawing fixed volume VOI/volume of interest (3D Sphere) over organs of interest namely basal ganglia (right and left), thalamus (right and left), cerebellum (right and left), parietal hemispheres (right and left), blood pool (right ventricle and left ventricle), liver (right and left lobe), spleen (upper and lower pole), pancreas (head and tail), renal cortex and renal pelvis (right and left). All the values hence obtained were decay corrected to account for variation in uptake period and time of acquisition by dividing sum of SUV values for appropriate region of interest by an appropriately computed Decay Factor as illustrated yielding % deposition indicating amount of DOPA uptake in the desired region of interest. The 18F-Dopa uptake in Brain & Different body organelles in for subject is provided in Table 8 below:

TABLE 8

| | | % Deposition | | | |
|---|---|---|---|---|---|
| Body Organ | | Placebo | Carbidopa | Example 1 | Example 2 |
| Basal | SUV Max | 4.62 | 5.90 | 6.11 | 5.40 |
| Ganglion | Avg | 0.79 | 1.28 | 1.36 | 1.46 |
| | Min | 0.07 | 0.06 | 0.15 | 0.33 |
| Thalamus | SUV Max | 1.82 | 3.16 | 3.05 | 2.27 |
| | Avg | 0.58 | 1.02 | 1.06 | 1.03 |
| | Min | 0.00 | 0.29 | 0.23 | 0.45 |
| Cerebellum | SUV Max | 1.71 | 3.77 | 2.66 | 2.16 |
| | Avg | 0.59 | 1.28 | 0.96 | 1.04 |
| | Min | 0.02 | 0.38 | 0.29 | 0.47 |
| Parietal | SUV Max | 1.67 | 3.29 | 3.98 | 2.12 |
| | Avg | 0.60 | 1.11 | 1.12 | 1.05 |
| | Min | 0.01 | 0.27 | 0.05 | 0.46 |
| Blood | SUV Max | 3.36 | 3.25 | 2.71 | 3.81 |
| | Avg | 0.78 | 1.31 | 1.21 | 1.23 |
| | Min | 0.06 | 0.36 | 0.61 | 0.51 |
| Liver | SUV Max | 2.51 | 2.73 | 2.94 | 2.96 |
| | Avg | 1.30 | 1.50 | 2.07 | 1.75 |
| | Min | 0.45 | 0.80 | 1.26 | 0.90 |
| Spleen | SUV Max | 2.20 | 2.67 | 2.11 | 2.65 |
| | Avg | 0.72 | 1.56 | 1.32 | 1.17 |
| | Min | 0.09 | 1.04 | 0.88 | 0.54 |
| Pancreas | SUV Max | 13.73 | 4.97 | 11.87 | 14.18 |
| | Avg | 6.27 | 2.18 | 8.38 | 6.72 |
| | Min | 1.90 | 0.72 | 5.08 | 2.35 |
| Renal Cortex | SUV Max | 6.08 | 5.28 | 5.89 | 6.53 |
| | Avg | 2.86 | 2.79 | 4.26 | 3.31 |
| | Min | 0.75 | 0.52 | 2.64 | 1.20 |
| PCS/Renal | | 0.79 | 0.82 | 0.82 | 0.85 |
| pelvis | Volume cm3 | 7.91 | 72.64 | 24.23 | 8.36 |
| | SUV Max | 3.91 | 16.27 | 8.18 | 4.36 |
| | Avg | 0.73 | 1.63 | 1.29 | 1.18 |
| | Min | 1.22 | 0.20 | 0.15 | 0.26 |

Table 8 demonstrates the results of the dopamine uptake to the brain and other body organelles in same human subjects with four different treatments i.e., Placebo, Example 1, Example 2 and Carbidopa. The dopamine % uptake in target points basal ganglion was as follows: 0.79 (Placebo), 1.28 (Carbidopa), 1.36 (Example 1) and 1.46 (Example 2).

It can be noted that the results for basal ganglia region (target organ of interest) indicated significant increase in DOPA tracer uptake using Carbidopa, Example 1 and Example 2 over placebo among all 6 subjects. Among the 2 drugs, Example 2 outperformed Example 1, and Carbidopa. Where, Example 1 results quite similar to Carbidopa group and outperformed placebo group in DOPA uptake. Hence, this finding could imply similar increments in Levodopa uptake in basal ganglia (caudate and putamen) which leads to subsequent activation of dopaminergic pathway resulting in better efficacy over commercially available Carbidopa and its analogues.

Similar to basal ganglia, thalamus (site of known extra striatal uptake of DOPA and significant role in planning and coordination of movement in body) also showed similar significant increment in DOPA tracer uptake using Carbidopa, Example 1 and Example 2 over placebo among all 6 subjects with Example 2 slightly outperforming Example 1 and providing similar increments 18F-DOPA uptake comparable and equivalent to Carbidopa.

In this regard cerebellar hemispheres and parietal cortex (Region of low/insignificant DOPA uptake and can be considered as background) also demonstrated similar pattern of increments and DOPA tracer uptake suggesting possible overall increased neuroparenchyma DOPA delivery due to peripheral DOPA decarboxylase suppression. Among whole body regions—blood pool, liver and spleen no obvious appreciable difference in DOPA uptake was appreciated among placebo, Carbidopa and Example 1 and Example 2 groups.

Further, having regards to pancreas (Organ with significant physiological DOPA uptake) significant reduction in DOPA uptake was observed with the use of Carbidopa as compared to placebo group. However, both Example 1 and Example 2 did not result in significant reduction in DOPA uptake suggesting possible differential mechanism of action/site of action or enzyme specificity like Carbidopa.

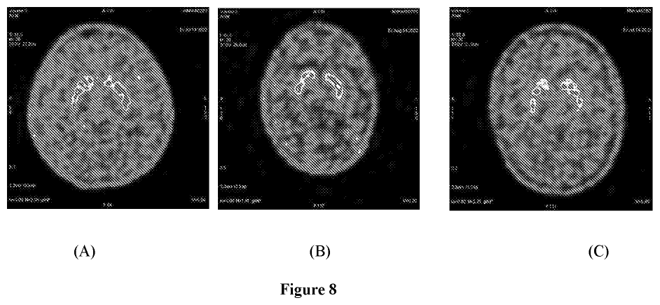

No significant differences were observed in renal cortical and renal pelvic calyceal system (PCS) DOPA uptake (representing partial mode of tracer excretion) Among Placebo, Carbidopa, Example 1 and Example 2 groups. PET images obtained from the study are provided as FIG. 7 and FIG. 8 further confirm the above conclusions.

The study concludes that composition according to the present disclosure were found to be 14.29% more effective in the subjects under study as compared to the established carbidopa composition. Further, the safety studies & the test drugs were found safe.

The study also indicated that the deposition of 18F-dopa in the peripheral circulation and deposition was found to be lowest for the inventive composition across different organs, when compared with carbidopa and placebo as referred in Table 8.

The foregoing description of the invention has been set merely to illustrate the invention and is not intended to be limiting. Since the modifications of the disclosed embodiments incorporating the spirit and substance of the invention may occur to the person skilled in the art, the invention should be construed to include everything within the scope of the disclosure.

1. A composition comprising an active ingredient or drug, one or more plant-derived neuroprotective ingredients and one or more nutraceutically or pharmaceutically acceptable excipient.

2. The composition as claimed in claim 1, wherein the active ingredient or drug is in the range of 10 mg to 500 mg, the plant-derived neuroprotective ingredients is in the range of 50 mg to 300 mg and the nutraceutically or pharmaceutically acceptable excipient is in the range of 1% to 20%.

3. The composition as claimed in claim 1, wherein the active ingredient or drug for degenerative disorders is selected form levodopa (L-DOPA), Avenanthramide, Chlorogenic acids, Cordycepin, Coriolin, Deoxycoriolic acid and cordycepic acid, Tetra hydro cannabinols, Cannabidiols or combination thereof.

4. The composition as claimed in claim 1, wherein the plant-derived neuroprotective ingredients is extracted from the plants belonging to the genus selected from vaccinium, brassica, allium, piper, curcuma, avena, mucuna, oats, green coffee, cannabis, cordyceps or mycelium.

5. The composition as claimed in claim 4, wherein the plant-derived neuroprotective ingredients are extracted from Mucuna pruriens, Avena sativa, Berberis aristata, Cordyceps militaris, Cordyceps sinensis, Vitis vinifera, Cannabis indica, Cannabis sativa, Coffea arabica, Curcuma longa, Piper nigrum, Allium cepa, Brassica oleracea and Vaccinium myrtillus.

6. The composition as claimed in claim 1, wherein the plant-derived neuroprotective ingredients are selected from the group consisting of, avenanthramides, berberine, cordycepin, polysaccharides, adenosine, resveratrol, phytochemicals, epigallocatechin gallate (EGCG), peperine, polyphenols preferably selected from curcumin or quercetin.

7. The composition as claimed in claim 1, wherein the drug is useful for degenerative disorders selected from Parkinson's disease (PD), Alzheimer, chronic depression, Transient ischemic stroke, Dementia, Epilepsy and Ataxia.

8. The composition as claimed in claim 1, wherein the nutraceutically or pharmaceutically acceptable excipient is selected from at least one diluent, at least one super disintegrant, at least one binder, at least one lubricant, at least one glidant and combinations thereof.

9. The composition as claimed in claim 1, in the form of a semi-solid mass, powder, an oil and water-soluble dispersion, nano emulsion, a capsule, tablet, syrup, a blend, a suspension, nasal drop or spray, dry powder, dermal patches, subcutaneous delivery or granules.

10. The composition as claimed in claim 1, comprising one or more active ingredients selected from vitamins, minerals, antioxidants, Omega, and trace elements and combinations thereof.

11. A method for preparing the composition claimed in claim 1, comprising the following steps:
proportioning and weighing the neuroprotective ingredients and converting into the powder;
sieving in through a pre-determined mesh size;
blending the superfine powder with the drug for degenerative disorders and one or more nutraceutically or pharmaceutically acceptable excipient;
filling the processed blend in the enteric coated capsule.

12. The method as claimed in claim 11, wherein the powder is a superfine powder.

13. The method as claimed in claim 11, wherein mesh size is in the range of 50 to 250.

14. The method as claimed in claim 11, wherein the method comprises one or more steps selected from wet granulation, direct compression, compressed coating, direct filling and wet coating.

15. A method for the treatment of a patient by delivering an active ingredient or drug for degenerative disorders, to a predetermined location of the body, comprising administering the composition as claimed in claim 1 in a patient in need of the said drug/active agent.

16. The method as claimed in claim 15, wherein the predetermined location of the body is basal ganglia.

17. Use of the composition as claimed in claim 1 for delivering an active ingredient or drug for degenerative disorders to a predetermined location in the body, preferably basal ganglia.

18. Use of the composition as claimed in claim 1 for improving the gut permeability.

19. Use of the composition as claimed in claim 1 for inhibiting the coagulation of α-synuclien protein.
